(19) United States
(12) Patent Application Publication
(10) Pub. No.: US 2025/0281562 A1
(43) Pub. Date: Sep. 11, 2025

(54) METHOD AND COMPOSITION FOR IMPROVING MUSCLE PERFORMANCE

(71) Applicants: NANJING NUTRABUILDING BIO-TECH CO., LTD., Nanjing (CN); AXIOM FOODS, INC., Los Angeles, CA (US)

(72) Inventors: Kylin LIAO, Nanjing (CN); Shawn WELLS, Nanjing (CN)

(21) Appl. No.: 19/213,174

(22) Filed: May 20, 2025

Related U.S. Application Data
(63) Continuation-in-part of application No. PCT/CN2023/088146, filed on Apr. 13, 2023.

(30) Foreign Application Priority Data
Nov. 21, 2022 (WO) PCT/CN2022/133252

(51) Int. Cl. A61K 38/01

(52) U.S. Cl. CPC A61K 38/011

(57) ABSTRACT

Disclosed herein is a method for improving muscle performance and/or improving exercise performance in a mammal, which comprises administering a composition to the mammal, the composition comprising an effective amount of a vegetable protein hydrolysate. Also disclosed is a composition comprising an effective amount of a vegetable protein hydrolysate and used for improving muscle performance and/or improving exercise performance in a mammal. The method and the composition of the present invention can ameliorate motion capability reduction caused by muscle injury, relieve muscle soreness, relieve or resist muscle injury, reduce inflammatory response, increase muscle mass, increase muscle strength and endurance; increase running and/or swimming distance, increase exercise time, increase running and/or swimming speed, improve exercise endurance or power, reduce inflammation and muscle fatigue after exercise, improve muscle contraction during exercise.

### CROSS REFERENCE TO RELATED APPLICATION

This application is a continuation-in-part application of International Patent Application No. PCT/CN2023/088146, filed on Apr. 13, 2023, which claims the priority of International Patent Application No. PCT/CN2022/133252, filed on Nov. 21, 2022, the contents of all of which are incorporated herein by reference in their entirety.

### FIELD OF THE INVENTION

This invention belongs to the technical field of dietary or nutritional supplements; specifically relates to a method and a composition for improving muscle performance and/or improving exercise performance in a mammal, especially ameliorating the decline of exercise ability caused by muscle injury, relieving muscle soreness, relieving or resisting muscle injury, reducing inflammatory response, increasing muscle mass, increasing muscle strength and endurance; increasing running and/or swimming distance, increasing exercise time, increasing running and/or swimming speed, improving exercise endurance or power, reducing inflammation and muscle fatigue after exercise, improving muscle contraction during exercise.

### BACKGROUND OF THE INVENTION

At present, people's demand for dietary or nutritional supplements is increasing, aiming at improving individual health and reducing disease risk. More often, athletes will use dietary or nutritional supplements to improve their strength and performance.

As we all know, the skeletal muscle injury caused by resistance-based exercise will promote the slight injury of muscle fibers, which may lead to temporary passive tension increase, delayed onset muscle soreness (DOMS) and the decrease of strength production, as well as the increase of intramuscular protein flowing into the blood. The degree of injury and discomfort may increase with the passage of time and last for a long time, especially for people who often do strenuous exercise or complete excessive exercise.

Protein based on a vegetable/plant, such as soybean, pea, wheat, rice, algae and hemp, has attracted considerable attention in the past few years. The growing demand for food and drinks with Clean Label, coupled with the increasing number of vegetarians in European countries mainly for environmental and health reasons, has promoted the demand for vegetable/plant-based protein. Clear vegetable/plant/vegan protein (CVP) is an innovative vegetable/plant-derived protein, such as derived from soybean, pea, rice, mung bean, hemp seed, etc.

Previous studies have proved that vegetable/plant-based protein has the functions of muscle building, anti-fatigue and improving exercise performance. However, the published research and comments did not provide a clear consensus that supplementing vegetable protein hydrolysate before, during or after eccentric resistance exercise can reduce muscle injury and accelerate the recovery of exercise performance. If a vegetable protein that can reduce muscle injury and accelerate the recovery of exercise performance can be extracted, it can tap and give full play to the resource potential of vegetable protein and provide help for human exercise and health.

### SUMMARY OF THE INVENTION

In one aspect, the invention provides a method for improving muscle performance and/or improving exercise performance in a mammal, comprising administering a composition to the mammal, the composition comprising an effective amount of a vegetable protein hydrolysate.

In some embodiments, the vegetable protein hydrolysate is selected from one or more of hydrolyzed pea protein, hydrolyzed soybean protein.

In some embodiments, the improving muscle performance includes ameliorating the decline of exercise ability caused by muscle injury, relieving muscle soreness, relieving or resisting muscle injury, reducing inflammatory response, increasing muscle mass, increasing muscle strength and endurance; the improving exercise performance includes one or more of the following: increasing running and/or swimming distance, increasing exercise time, increasing running and/or swimming speed, improving exercise endurance or power, reducing inflammation and muscle fatigue after exercise, improving muscle contraction during exercise.

In some embodiments, the ameliorating the decline of exercise ability caused by muscle injury includes improving bench press ability, vertical jump height, and legs and back strength; the relieving muscle soreness includes relieving delayed onset muscle soreness; the relieving or resisting muscle injury includes reducing or controlling serum myoglobin, creatine kinase; the reducing inflammatory response includes reducing or controlling one or more inflammatory factors: IL-1α, IL-1β, TNF-α, IL-6, C-reactive protein.

In some embodiments, the composition is prepared in a form of food, drink, nutritional supplement, animal feed, or pharmaceutical composition.

In some embodiments, the vegetable protein hydrolysate is administrated at a daily dose of 0.1-500 g. In some embodiments, the vegetable protein hydrolysate is administrated at a daily dose of 0.1-500 g, 1-400 g, 2-300 g, 3-200 g, 5-100 g.

In some embodiments, the vegetable protein hydrolysate is formulated in solutions, liquid suspensions, parenteral solutions, injections, tablets, pills, granules, powders, film, (micro) capsules, aerosols, tonics, syrups, beverages, nourishments, snacks, bars, gums, sugars, or functionalized food compositions.

In some embodiments, the administration is through various routes selected from oral administration, intravenous injection, intramuscular injection, intraperitoneal injection, or sublingual administration.

In some embodiments, the composition is administered before, during or after exercise.

In another aspect, the invention provides a composition comprising an effective amount of a vegetable protein hydrolysate, for improving muscle performance and/or improving exercise performance in a mammal.

In some embodiments, the vegetable protein hydrolysate is selected from one or more of hydrolyzed pea protein, hydrolyzed soybean protein.

In some embodiments, the improving muscle performance includes ameliorating the decline of exercise ability caused by muscle injury, relieving muscle soreness, relieving or resisting muscle injury, reducing inflammatory response, increasing muscle mass, increasing muscle strength and endurance; the improving exercise performance includes one or more of the following: increasing running and/or swimming distance, increasing exercise time, increasing running and/or swimming speed, improving exercise endurance or power, reducing inflammation and muscle fatigue after exercise, improving muscle contraction during exercise.

In some embodiments, the ameliorating the decline of exercise ability caused by muscle injury includes improving bench press ability, vertical jump height, and legs and back strength; the relieving muscle soreness includes relieving delayed onset muscle soreness; the relieving or resisting muscle injury includes reducing or controlling serum myoglobin, creatine kinase; the reducing inflammatory response includes reducing or controlling one or more inflammatory factors: IL-1a, IL-1B, TNF-α, IL-6, C-reactive protein.

In some embodiments, the composition is prepared in a form of food, drink, nutritional supplement, animal feed, or pharmaceutical composition.

In some embodiments, the vegetable protein hydrolysate is administrated at a daily dose of 0.1-500 g. In some embodiments, the vegetable protein hydrolysate is administrated at a daily dose of 0.1-500 g, 1-400 g, 2-300 g, 3-200 g, 5-100 g.

In some embodiments, the vegetable protein hydrolysate is formulated in solutions, liquid suspensions, parenteral solutions, injections, tablets, pills, granules, powders, film, (micro) capsules, aerosols, tonics, syrups, beverages, nourishments, snacks, bars, gums, sugars, or functionalized food compositions.

In another aspect, the invention provides use of a composition for preparing food, drink, nutritional supplement, animal feed, or pharmaceutical composition for improving muscle performance and/or improving exercise performance in a mammal, wherein the composition comprises an effective amount of a vegetable protein hydrolysate.

In some embodiments, the vegetable protein hydrolysate is selected from one or more of hydrolyzed pea protein, hydrolyzed soybean protein.

In some embodiments, the improving muscle performance includes ameliorating the decline of exercise ability caused by muscle injury, relieving muscle soreness, relieving or resisting muscle injury, reducing inflammatory response, increasing muscle mass, increasing muscle strength and endurance; the improving exercise performance includes one or more of the following: increasing running and/or swimming distance, increasing exercise time, increasing running and/or swimming speed, improving exercise endurance or power, reducing inflammation and muscle fatigue after exercise, improving muscle contraction during exercise.

In some embodiments, the ameliorating the decline of exercise ability caused by muscle injury includes improving bench press ability, vertical jump height, and legs and back strength; the relieving muscle soreness includes relieving delayed onset muscle soreness; the relieving or resisting muscle injury includes reducing or controlling serum myoglobin, creatine kinase; the reducing inflammatory response includes reducing or controlling one or more inflammatory factors: IL-1α, IL-1β, TNF-α, IL-6, C-reactive protein.

In some embodiments, the vegetable protein hydrolysate is administrated at a daily dose of 0.1-500 g. In some embodiments, the vegetable protein hydrolysate is administrated at a daily dose of 0.1-500 g, 1-400 g, 2-300 g, 3-200 g, 5-100 g.

In some embodiments, the vegetable protein hydrolysate is formulated in solutions, liquid suspensions, parenteral solutions, injections, tablets, pills, granules, powders, film, (micro) capsules, aerosols, tonics, syrups, beverages, nourishments, snacks, bars, gums, sugars, or functionalized food compositions.

Compared with the prior art, the composition and method of the present invention can improve muscle performance and/or improve exercise performance, especially ameliorate the decline of exercise ability caused by muscle injury, relieve muscle soreness, relieve or resist muscle injury, reduce inflammatory response, increase muscle mass, increase muscle strength and endurance; increase running and/or swimming distance, increase exercise time, increase running and/or swimming speed, improve exercise endurance or power, reduce inflammation and muscle fatigue after exercise, improve muscle contraction during exercise, and provide help for human exercise and health. In addition, the source of vegetable protein hydrolysate is green and environmentally friendly, which can give full play to the resource potential of vegetable protein.

### BRIEF DESCRIPTION OF THE FIGURES

FIG. 1 shows the changes of delayed onset muscle soreness (DOMS) scores of three groups of subjects after squat exercise.

FIG. 2 shows the changes of serum myoglobin concentration in three groups of subjects.

FIG. 3 shows the changes of serum creatine kinase (CK) concentration in three groups of subjects.

FIG. 4a shows the changes of TNF-α levels in three groups of subjects.

FIG. 4b shows the changes of IL-6 levels in three groups of subjects.

FIG. 4c shows the changes of C-reactive protein (CRP) levels in three groups of subjects.

### DETAILED DESCRPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As use herein, the terms “about” and “approximately” provide numerical flexibility by providing endpoints where a given value can be “slightly above” or “less than”. The flexibility of this term can be determined by specific variables and based on experience and related descriptions herein within the knowledge of those skilled in the art.

As used herein, the term “effective amount” refers to the amount required to achieve the effect as taught herein. The specific effective dose level for any particular subject will depend upon a variety of factors including the signs being treated and the severity of the signs; the specific composition employed; the age, body weight, general health, sex and diet of the subject; the time of administration, route of administration, and rate of excretion of vegetable protein hydrolysate employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed compositions, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of vegetable protein hydrolysate and/or composition comprising the same may range broadly, depending upon the desired effects and the indication. The daily dosage regimen for an adult human patient may be, for example, an oral dose of between 0.1-10 g of vegetable protein hydrolysate, preferably 0.2-8 g, 0.3-6 g, 0.5-5 g, or 1-3 g vegetable protein hydrolysate per kg of body weight of the subject. The dosage may be a single one or a series of two or more given in the course of one or more days, as is needed by the subject. In some embodiments, the compounds are administered for a period of time, for example for a week or more, or for months or years.

As used herein, the term “administration” refers to the process of delivering a disclosed composition or active ingredient to a subject. The compositions of the invention can be administered in a variety of suitable ways, including orally, intragastrically, and parenterally (e.g., intravenous and intraarterial as well as other suitable parenteral routes), and the like, to exert the desired effects.

Multiple techniques of administering a composition exist in the art including, but not limited to, oral, rectal, topical, aerosol, injection and parenteral delivery, including intramuscular, subcutaneous, intravenous, intramedullary injections, intrathecal, intraventricular, intraperitoneal, intranasal and intraocular injections.

In some embodiments, the vegetable protein hydrolysate and/or the composition comprising the same of the present invention can be prepared into a composition together with a dietetically or pharmaceutically acceptable carrier. The above carriers include non-toxic and compatible substances commonly used in health foods, dietary supplements and pharmaceutical preparations, such as sugar, starch, cellulose and its derivatives, powdered tragacanth, malt, gelatin, talc, oil, diol, polyol, ester, agar, alginic acid, pyrogen-free water, isotonic saline, etc.

In some embodiments, the vegetable protein hydrolysate and/or the composition comprising the same of the present invention can be administered together with other supplements, such as vitamins, minerals, tonics and other supplements known in the art.

In some embodiments, the vegetable protein hydrolysate and/or the composition comprising the same of the present invention may be provided as a solid or powder form. The compositions in such solid form may be formulated so as to provide for sufficient ease of handling and manufacturability. The compositions may be provided as a liquid, such as in the form of a shot or mouth spray for fast delivery and absorption. Liquid forms may include one or more liquid carriers, such as water, ethanol, glycerin, propylene glycol, 1,3-propandiol, and the like

In some embodiments, the compositions of the present invention can be used as suppository, tablet, pill, granule, powder, film, capsule, beverage, aerosol, alcohol, tincture, tonic, liquid suspension or syrup.

The following examples are illustrative of select embodiments of the present invention and are not meant to limit the scope of the invention.

### EXAMPLES

Fifteen young men (age 22.3±1.5 years, height 175.4±6.7 cm, weight 86.4±15.6 kg) with several years (5.3±2.5 years) of resistance training experience and self-reported average training time of 7.3±2.1 hours per week were randomly divided into three groups: pea protein hydrolysate group (PPH), soybean protein hydrolysate group (SPH), and pure water group (Water). Subjects were required not to take additional protein supplements (such as whey, casein) during the study.

During the 8-day study, unless otherwise specified, the subjects engaged in normal physical activities and food intake. PPH group was administered 20 g hydrolyzed pea protein dissolved in 250 ml water; SPH group was administered 20 g hydrolyzed soybean protein dissolved in 250 ml water; pure water group was administered 250 ml of pure water, and each group was treated double-blind in shake flask.

On the first day, the serum indexes (baseline) of the subjects in fasting state were detected, and then the three groups were administered hydrolyzed pea protein, hydrolyzed soybean protein or pure water respectively, and then three physical fitness tests (baseline) were carried out. The three groups were administered only hydrolyzed pea protein, hydrolyzed soybean protein or pure water respectively every morning from day 2 to day 4. On the morning of day 5 (training day), the subjects did squats in fasting state, and immediately took hydrolyzed pea protein, hydrolyzed soybean protein or pure water respectively after exercise. Serum indexes were detected at 4, 24, 48 and 72 hours after exercise, and delayed onset muscle soreness (DOMS) scale was filled out at 1, 2, 4, 24, 48 and 72 hours after exercise. On the morning of day 6, day 7 and day 8 (24, 48 and 72 hours after exercise, respectively), serum indexes were detected and delayed onset muscle soreness (DOMS) scale was filled out in fasting state in the subjects. Then hydrolyzed pea protein, hydrolyzed soybean protein or pure water was administered to each group respectively, and then three physical fitness tests were carried out.

### Example 1 Physical Fitness Test

The bench press ability, vertical jump height, and leg and back strength of the subjects were tested. Bench press is performed until subjects are exhausted, subjects lie flat on bench, and press barbell equivalent to 75% of their own weight on the bench at a lifting speed of 30 times per minute as many times as possible. In the vertical jump test, subjects jump as high as possible and tap the measuring equipment with one hand to record the difference between the height of high jump and the standing height; repeat the test three times and record the best score. Legs/lower back strength is evaluated by a dynamometer: with a slight bend at the knee, subject stretches out his arm and grabs the rod connected to the dynamometer through a chain, and then lifts it with maximum strength for a few seconds; repeat the test three times and record the best score.

The physical fitness test results are shown in the following table:

| Item | Group | Baseline | Day 6 | Day 7 | Day 8 |
|---|---|---|---|---|---|
| Bench press | SPH | 13.6 | 11.5 | 12.8 | 13.7 |
| (number) | PPH | 12.8 | 11.1 | 12.2 | 12.7 |
| | Water | 12.5 | 7.8 | 8.5 | 10.1 |
| Vertical jump | SPH | 62.3 | 49.1 | 53.6 | 58.4 |
| (cm) | PPH | 62.5 | 47.5 | 50.9 | 53.5 |
| | Water | 62.8 | 46.6 | 46.8 | 49.5 |
| Legs and back | SPH | 1.56 | 1.47 | 1.55 | 1.61 |
| strength | PPH | 1.58 | 1.46 | 1.53 | 1.6 |
| (kg/kg BW) | Water | 1.56 | 1.42 | 1.47 | 1.55 |

As can be seen from the table, squat exercise can lead to muscle injury, and on the sixth day, the level of three physical fitness tests in each group decreased greatly. However, with the passage of time, the bench press ability of SPH group and PPH group was obviously improved, and it had returned to the baseline level by the eighth day. The vertical jump height of SPH group and PPH group was greatly improved on the 7th and 8th day, respectively, which was significantly higher than that of pure water group. The legs and back strength of each group gradually recovered over time, and by the eighth day, the legs and back strength of SPH group and PPH group was even greater than the baseline level. It shows that the decline of exercise ability caused by muscle injury is ameliorated in CVP groups, and the effect in SPH group is more obvious.

### Example 2 Muscle Soreness Index Score

The subjects performed three groups of squats, with 20 squats in each group and a rest of 30 seconds between each group. This eccentric exercise (squat) can effectively induce muscle injury.

Subjects were asked to use VAS scale to evaluate the degree of muscle soreness they felt. At each time point (1, 2, 4, 24, 48 and 72 hours after squat exercise), the degree of pain was evaluated by drawing a vertical continuous line from 0 to 10 cm (0 cm=no pain, 10 cm=extreme pain). The evaluation method of pain degree is to measure the distance of each mark from 0 to the nearest tenth centimeter.

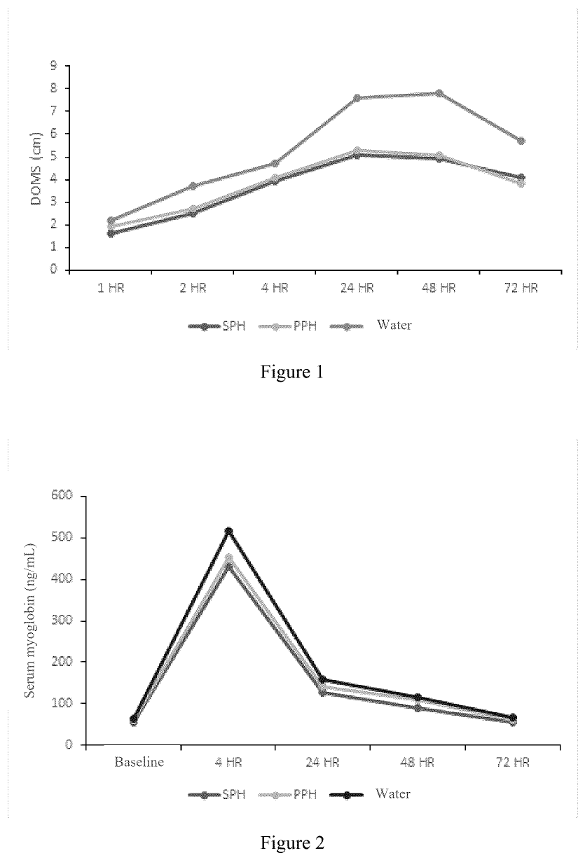

FIG. 1 shows the changes of delayed onset muscle soreness (DOMS) scores of three groups of subjects after squat exercise. As shown in FIG. 1, the exercise scheme leads to DOMS in all groups. The VAS scores of SPH group and PPH group reached the peak at 24 hours after exercise, and then began to decline. The VAS score of pure water group reached the peak at 48 hours, and then began to decline. And at each time point, the VAS scores of SPH group and PPH group were significantly lower than those of pure water group. It shows that CVP can effectively relieve muscle soreness after exercise.

### Example 3 Evaluation of Serum Biochemical Indexes

The primary outcome indexes of blood sample analysis are serum muscle injury biomarkers, and the secondary outcome indexes are inflammatory indexes. Blood samples were collected in a serum separation tube and centrifuged. Serum myoglobin, creatine kinase (CK), inflammatory factors TNF-α and IL-6, C-reactive protein (CRP) and comprehensive diagnostic chemistry were analyzed with corresponding detection kits.

FIG. 2 shows the changes of serum myoglobin concentration in three groups of subjects. As shown in FIG. 2, the concentration of serum myoglobin reached a peak 4 hours after squat exercise, and then decreased. The concentration of serum myoglobin in SPH group and PPH group was lower than that in pure water group at every time point, especially lower in SPH group. The concentration of serum myoglobin in CVP groups decreased, which indicated that CVP could relieve muscle injury. It shows that CVP can effectively reduce or control serum myoglobin and relieve muscle injury after exercise.

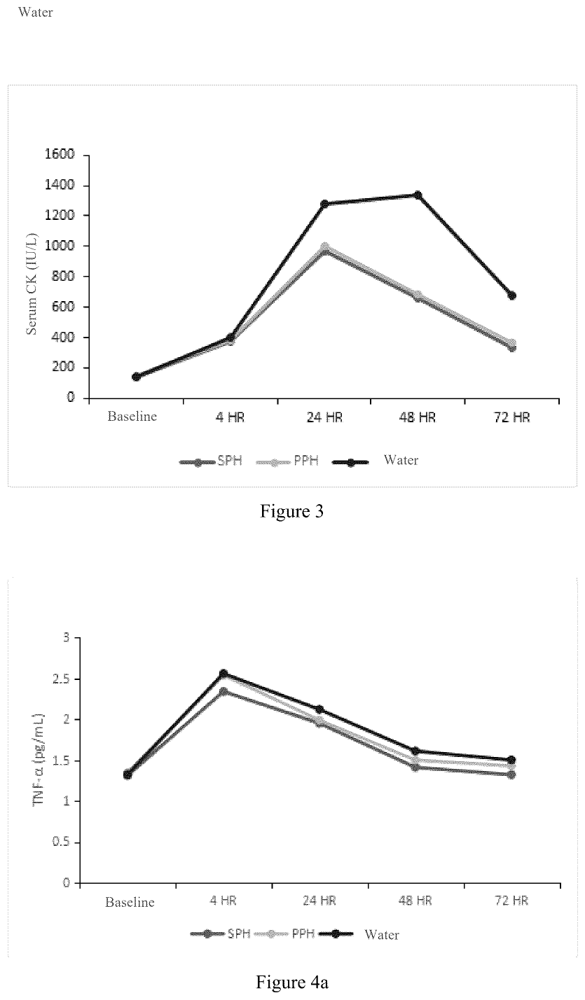

FIG. 3 shows the changes of serum creatine kinase (CK) concentration in three groups of subjects. As shown in FIG. 3, the CK concentration in SPH group and PPH group reached a peak 24 hours after squat exercise, and then began to decrease significantly. The CK concentration in pure water group increased greatly from 4 hours to 24 hours, reached a peak at 48 hours, and then began to decline. And at each time point, the CK concentration in SPH group and PPH group was obviously lower than that in pure water group. It shows that CVP can effectively reduce or control serum CK and relieve muscle injury after exercise.

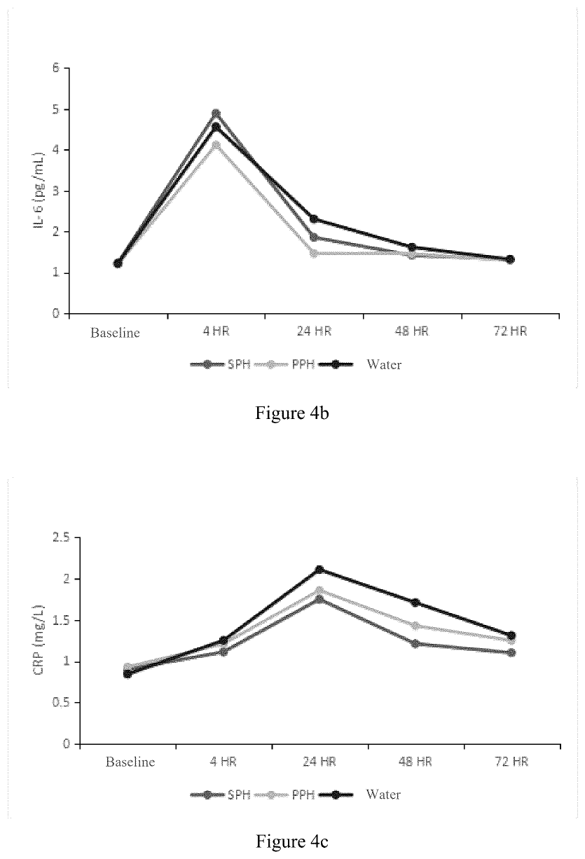

FIGS. 4a-4b show the changes of levels of serum inflammatory factors. FIG. 4a shows the changes of TNF-α levels in three groups of subjects. As shown in FIG. 4a, the TNF-α of the three groups of subjects reached a peak 4 hours after exercise, and the TNF-α levels in SPH group and PPH group were all lower than those in pure water group at 24, 48 and 72 hours after exercise. FIG. 4b shows the changes of IL-6 levels in three groups of subjects. As shown in FIG. 4b, the IL-6 of the three groups of subjects reached a peak 4 hours after exercise. Although the IL-6 level in SPH group was slightly higher at 4 hours after exercise, it became obviously lower than that in pure water group at 24 hours after exercise, like in PPH group. FIG. 4c shows the changes of C-reactive protein (CRP) levels in three groups of subjects. As shown in FIG. 4c, the CRP levels of the three groups of subjects all reached a peak 24 hours after squat exercise, and then decreased. The CRP levels in SPH group and PPH group were all obviously lower than those in pure water group at each time point after squat exercise. After exercise, the concentrations of serum inflammatory factors in clear vegetable protein (CVP) groups were all lower than those in pure water group. It shows that CVP can effectively reduce or control inflammatory factors.

In conclusion, CVP can ameliorate the decline of exercise ability caused by muscle injury, improve exercise performance; effectively relieve muscle soreness after exercise, such as DOMS; effectively relieve or resist muscle injury after exercise; effectively reduce or control the levels of inflammatory factors, reduce inflammatory response; effectively increase muscle mass, effectively increase muscle strength and endurance; effectively increase running and/or swimming distance, effectively increase exercise time, effectively increase running and/or swimming speed; effectively improve exercise endurance or power; effectively reduce inflammation and muscle fatigue after exercise, effectively improve muscle contraction during exercise.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. A method for improving muscle performance and/or improving exercise performance in a mammal, comprising administering a composition to the mammal, the composition comprising an effective amount of a vegetable protein hydrolysate.

2. The method of claim 1, wherein the vegetable protein hydrolysate is selected from one or more of hydrolyzed pea protein, hydrolyzed soybean protein.

3. The method of claim 1, wherein the improving muscle performance includes ameliorating the decline of exercise ability caused by muscle injury, relieving muscle soreness, relieving or resisting muscle injury, reducing inflammatory response, increasing muscle mass, increasing muscle strength and endurance; the improving exercise performance includes one or more of the following: increasing running and/or swimming distance, increasing exercise time, increasing running and/or swimming speed, improving exercise endurance or power, reducing inflammation and muscle fatigue after exercise, improving muscle contraction during exercise.

4. The method of claim 3, wherein the ameliorating the decline of exercise ability caused by muscle injury includes improving bench press ability, vertical jump height, and legs and back strength; the relieving muscle soreness includes relieving delayed onset muscle soreness; the relieving or resisting muscle injury includes reducing or controlling serum myoglobin, creatine kinase; the reducing inflammatory response includes reducing or controlling one or more inflammatory factors: IL-1a, IL-1B, TNF-α, IL-6, C-reactive protein.

5. The method of claim 1, wherein the composition is prepared in a form of food, drink, nutritional supplement, animal feed, or pharmaceutical composition.

6. The method of claim 1, wherein the vegetable protein hydrolysate is administrated at a daily dose of 0.1-500 g.

7. The method of claim 1, wherein the vegetable protein hydrolysate is formulated in solutions, liquid suspensions, parenteral solutions, injections, tablets, pills, granules, powders, film, (micro) capsules, aerosols, tonics, syrups, beverages, nourishments, snacks, bars, gums, sugars, or functionalized food compositions.

8. The method of claim 1, wherein the administration is through various routes selected from oral administration, intravenous injection, intramuscular injection, intraperitoneal injection, or sublingual administration.

9. The method of claim 1, wherein the composition is administered before, during or after exercise.
